(19) United States
(12) Patent Application Publication
(10) Pub. No.: US 2024/0368075 A1
(43) Pub. Date: Nov. 7, 2024

(54) Synthesis of N,N-Dialkyl, -Dialkenyl, -Dialkynyl, and Related Cyclics, Sulfamoyl Fluoride Compounds Using Hydrogen Fluoride

(71) Applicant: SES Holdings Pte. Ltd., Singapore (SG)

(72) Inventors: Rajendra P. Singh, Woburn, MA (US); Qichao Hu, Arlington, MA (US)

(21) Appl. No.: 18/535,114

(22) PCT Filed: Oct. 29, 2021
(86) PCT No.: PCT/IB2021/060033

§ 371 Date: Dec. 11, 2023

Related U.S. Application Data
(60) Provisional application No. 63/217,968, filed on Jul. 2, 2021.

(51) Int. Cl. C07C 307/02; B01J 23/18; B01J 27/12

(52) U.S. Cl. CPC C07C 307/02; B01J 23/18; B01J 27/12

(57) ABSTRACT

Methods of producing N,N-dimethyl sulfamoyl fluoride and related derivatives of the formula F—S(O)₂—NR₂ (I) by contacting a sulfamoyl nonfluorohalide compound of the formula X—S(O)₂—NR₂ (II) with anhydrous hydrogen fluoride under conditions sufficient to produce the N,N-dimethyl sulfamoyl fluoride or derivative thereof of Formula I, wherein R in each of Formulas I and II is, independently, a linear or branched alkyl, alkenyl, or alkynyl group containing 1 to 12 carbon atoms, the Rs can be joined to form a cyclic amine with the N, and X is any one of chlorine, bromine, and iodine.

### RELATED APPLICATION DATA

This application claims the benefit of priority to U.S. Provisional Patent Application Ser. No. 63/217,968, filed Jul. 2, 2021, and titled “SYNTHESIS OF N,N-DIALKYL-ARYL, OR-CYCLOALKYL SULFAMOYL FLUORIDE USING HYDROGEN FLUORIDE”, which is incorporated by reference herein in its entirety.

### FIELD OF THE INVENTION

The present invention relates to synthesis of sulfamoyl fluoride compounds. More particularly, the present invention is directed to synthesis of N,N-dialkyl-dialkenyl, -dialkynyl, and related cyclics, sulfamoyl fluoride compounds using hydrogen fluoride.

### BACKGROUND OF THE INVENTION

Incorporation of fluorine into molecules often results in a significant change in the physical and chemical properties of the molecules. Some fluorine-containing compounds have high electrochemical stability and are useful in electrochemical energy storage devices such as batteries and electric double layer capacitors and in biological field.

The compound N-(fluorosulfonyl) dimethylamine (FSO₂NMe₂) has been proposed as a solvent or additive for lithium-ion batteries (Chinese Patent No. CN 1 289 765A). At present, FSO₂NMe₂ is not commercially available in large amounts due to synthesis difficulties.

FSO₂NMe₂ was first prepared in the 1930s by metathesis between N,N-dimethyl sulfamoyl chloride (ClSO₂NMe₂) and potassium, sodium, or zinc fluoride in water (French Patent No. FR 806 383; German Patent No. DE 667 544; U.S. Pat. No. 2,130,038).

The reaction of SO₂F₂ with a secondary amine was first performed in 1948 (Emeléus, H. J., Wood, J. F., Journal of the Chemical Society (Resumed), 1948, 2183-2188). In this paper, diethylamine (Et₂NH) was dropped into a cooled (−78° C.) solution of SO₂F₂ in ethyl ether, and the product, FSO₂NEt₂, was obtained in a yield of 35%.

FSO₂NMe₂ has also been prepared by the reaction of N,N-dimethylaminosulfamide (Me₂NSO₂NH₂) with fluorosulfonyl isocyanate (FSO₂N═C═O) at 80° C. (Appel, R.; Montenarh, M., Chemische Berichte, 1977, 110, 2368-2373).

The reaction of SO₂F₂ with piperidine (HN(CH₂)₅) was performed in 1982 (Padma, D. K., Subrahmanya Bhat, V., Vasudeva Murthy, A. R., Journal of Fluorine Chemistry, 1982, 20, 425-437). SO₂F₂ was added into piperidine in ether at liquid nitrogen temperatures, followed by warming. Either FSO₂N(CH₂)₅ or SO₂(N(CH₂)₅)₂ was obtained depending on the amount of piperidine used.

Synthesis of N,N-dimethyl sulfamoyl fluoride (“DMSF”) is also reported by reaction of dimethylamine hydrochloride with gaseous sulfuryl fluoride. In this process, mixtures of products are formed and are hard to separate from the desired/indicated product. Due to these drawbacks, scaling-up of this process for commercial DMSF production is not economical.

Accordingly, there is a need for less-costly methods for producing high purity N,N-dimethyl sulfamoyl fluoride compounds and derivatives thereof, especially at commercial scale.

### SUMMARY OF THE DISCLOSURE

In an implementation, the present disclosure is directed to a method of producing a sulfamoyl fluoride compound of the formula F—SO₂—NR₂, wherein either 1) each R is, independently, a linear or branched alkyl, alkenyl, or alkynyl group containing 1 to 12 carbon atoms or 2) R₂ forms a cyclic amine with the N. The method includes adding a sulfamoyl nonfluorohalide of the formula X—SO₂—NR₂ and hydrogen fluoride (HF) to a reaction chamber of a reaction apparatus, wherein X is selected from the group consisting of chlorine (CI), bromine (Br), and iodine (I); providing conditions sufficient to support a reaction between the sulfamoyl nonfluorohalide and the HF that forms the sulfamoyl fluoride compound and an HX byproduct; and selectively removing at least some of the HX byproduct so as to yield the sulfamoyl fluoride compound.

### BRIEF DESCRIPTION OF THE DRAWINGS

For the purpose of illustration, the drawings show depictions of one or more aspects of the present disclosure. However, it should be understood that the depicted aspects are not limited to the precise arrangements and instrumentalities shown in the drawings, wherein:

FIG. 1 is diagram illustrating an example process for synthesizing a sulfamoyl fluoride product of the present disclosure, using N,N-dimethyl sulfamoyl fluoride (DMSF) as the demonstrative sulfamoyl fluoride product; and

FIG. 2 is diagram illustrating another example process for synthesizing a sulfamoyl fluoride product of the present disclosure, using DMSF as the demonstrative sulfamoyl fluoride product.

### DETAILED DESCRIPTION

In some aspects, the present disclosure describes methods of producing N,N-dimethyl sulfamoyl fluoride and related derivatives of the formula F—S(O)₂—NR₂ (I) by contacting a sulfamoyl nonfluorohalide compound of the formula X—S(O)₂—NR₂ (II) with anhydrous hydrogen fluoride under conditions sufficient to produce the N,N-dimethyl sulfamoyl fluoride or derivative thereof of Formula I, wherein R in each of Formulas I and II is, independently, a linear or branched alkyl, alkenyl, or alkynyl group containing 1 to 12 carbon atoms (e.g., a methyl, ethyl, propyl, or aryl group, among others), the Rs can be joined to form a cyclic amine with the N, and X is any one of chlorine, bromine, and iodine.

### Definitions

For the sake of this disclosure and the appended claims, the following definitions are used to increase clarity of the scope of the present inventions.

“Alkyl” refers to a saturated linear monovalent hydrocarbon moiety of one to twelve, typically one to six, carbon atoms or a saturated branched monovalent hydrocarbon moiety of three to twelve, typically three to six, carbon atoms. Alkyl groups can be optionally substituted with an alkoxide (i.e., —OR^{a}, wherein R^{a} is alkyl) and/or other functional group(s) that are either protected or non-reactive under a given reaction condition.

“Alkenyl” means a linear monovalent hydrocarbon moiety of two to twelve, typically two to six carbon atoms or a branched monovalent hydrocarbon moiety of three to twelve, typically three to six carbon atoms, containing at least one carbon-carbon double bond. Alkenyl groups can optionally be substituted with one or more functional groups that are either protected or non-reactive under a given reaction condition. Exemplary alkenyl groups include, but are not limited to, ethenyl, propenyl, butenyl, and the like.

“Alkynyl” means a linear monovalent hydrocarbon moiety of two to twelve, typically two to six carbon atoms or a branched monovalent hydrocarbon moiety of three to twelve, typically three to six carbon atoms, containing at least one carbon-carbon triple bond. Alkynyl groups can optionally be substituted with one or more functional groups that are either protected or non-reactive under a given reaction condition. Exemplary alkynyl groups include, but are not limited to, ethynyl, propynyl, butynyl, and the like.

The terms “halo”, “halogen”, and “halide” are used interchangeably and refer to fluoro, chloro, bromo, or iodo compounds or fluorine, chlorine, bromine, or iodine atoms according to the usage context.

The terms “nonfluorohalide”, “nonfluorohalo”, and “nonfluorohalogen” are used interchangeable and refer to chloro, bromo, or iodo compounds or chlorine, bromine, or iodine atoms according to the usage context.

The term “optionally substituted” means that the group is optionally substituted with one or more substituents that are nonreactive under a given reaction condition.

When describing a chemical reaction, the terms “treating”, “contacting”, and “reacting” are used interchangeably and refer to adding or mixing two or more reagents under appropriate conditions to produce the indicated and/or the desired product. It should be appreciated that the reaction that produces the indicated and/or the desired product may not necessarily result directly from the combination of two reagents that were initially added, i.e., there may be one or more intermediates that are produced in the mixture that ultimately lead to the formation of the indicated and/or the desired product.

The term “about” when used with a corresponding numeric value or other quantitative measure refers to ±20% of the numeric value, typically ±10% of the numeric value, often ±5% of the numeric value, and most often ±2% of the numeric value. In some embodiments, the term “about” can mean the numeric value itself.

### General

N,N-dimethyl sulfamoyl fluoride (DMSF; (CH₃)₂NSO₂F) and related derivatives are useful in various applications, including as solvents in electrolytes for electrochemical devices such as batteries and supercapacitors. Aspects of the present disclosure are directed to the synthesis of DMSF and related derivatives, which are useful solvents in batteries, including lithium-ion batteries and lithium-metal batteries. DMSF is also used as an intermediate in synthesizing medicinal compounds. DMSF is hydrolytically stable and has capacity to form, for example, a lithium fluoride (LiF) solid-electrolyte interphase (SEI) layer in lithium metal batteries.

The present inventors have recently disclosed the synthesis of DMSF using bismuth trifluoride (BiF₃) as a fluorinating reagent and converting N,N-dimethylsulfamoyl chloride (DMSCl; (CH₃)₂NSO₂Cl) to DMSF in excellent yield. This chemistry involves a liquid and solid reactant and produces a liquid desired/indicated product and BiCl₃ as a solid byproduct.

In contrast, the present disclosure presents processes of making DMSF, or related derivative, using anhydrous HF as a fluorination reagent and converting, quantitatively, a precursor N,N-dialkyl sulfamoyl nonfluorohalide or related precursor sulfamoyl nonfluorohalide into DMSF or related derivative. These new processes are economical, since it is easy to operate a continuous process due to both reactants being liquid below 25° C., which is not the case with the abovementioned synthesis process using BiF₃.

Under proper conditions, processes of the present disclosure are able to achieve a substantially higher yield of DMSF (or related derivative) by reacting N,N-dimethyl sulfamoyl chloride (or related precursor sulfamoyl nonfluorohalide to the desired/indicated derivative) with hydrogen fluoride. In some embodiments, the reaction also produces hydrogen chloride (HCl). In some instances, the step of reacting DMSCl (or other precursor sulfamoyl nonfluorohalide corresponding to the desired/indicated derivative) with HF also comprises removing HCl (or other hydrogen nonfluorohalide) that is produced in the reaction. In the specific example of HCl, the boiling point of HCl is lower than the boiling point of HF added. Therefore, HCl can be removed by simple distillation or evaporation. Any HF that may distill or evaporate during the process of removing HCl can be condensed and returned back into the reaction mixture. Generally, by adjusting the condensation temperature, the HF can be selectively condensed while allowing HCl to be distilled away from the reaction mixture. HCl can also be captured by passing reaction vapor through another condenser at a temperature that is sufficiently low enough to allow HCl to be captured. Alternatively, HCl can be neutralized by contacting with a base. In another method, HCl can be captured in water to yield an aqueous acid. Those skilled in the art will understand how to remove hydrogen halogenides other than HCl, such that descriptions of those are not necessary herein for them to implement the present invention to its fullest scope.

By conducting the reaction at an ambient (e.g., atmospheric) pressure condition, the present inventors have discovered that high yields of DMSF (or the desired/indicated related derivative) can be produced using HF. By removing HCl (or other hydrogen nonfluorohalide) as it is generated during the reaction further increases the yield of DMSF in accordance with Le Chatelier's Principle.

Methods of the present disclosure can be carried out by adding HF batchwise. Typically, the addition of HF is done with the HF in gaseous form, and the HF is allowed to condense back into the reaction mixture via a condenser. Alternatively, the reaction can be conducted by adding HF continually or continuously until a desired amount of HF has been added. Still alternatively, HF can be added substantially all at once, as fast as the desired amount of HF condensation can be achieved. Typically, however, HF is continuously added or added in a controlled manner throughout the reaction time at a constant temperature.

The amount of HF added to the reaction is at least about 1 equivalent compared to the amount of DMSF (or the desired/indicated related derivative) added. It should be appreciated that theoretically 1 mole of DMSCl (or other precursor sulfamoyl nonfluorohalide to the desired/indicated related derivative) requires 1 mole of HF to produce the desired/indicated DMSF (or the desired/indicated related derivative). Accordingly, 1 equivalent of HF is equal to the number of moles of DMSCl (or other precursor sulfamoyl nonfluorohalide to the desired/indicated related derivative) used. For example, if 1 mole of DMSCl (or other precursor sulfamoyl nonfluorohalide to the desired/indicated related derivative) is used, then 1 equivalent of HF is 1 mole of HF. Because there can be some loss of HF in the reaction, typically, but not necessarily, the total amount of HF added may be more than 1 equivalent, often at least about 1.5 equivalents, more often at least about 2 equivalents, and still more often at least about 2.5 equivalents.

Typically, the reaction temperature is at least about 20° C., often at least about 60° C., more often at least about 90° C., and at times at least about 100° C. The present inventors have found that under certain reaction conditions reacting HF with DMSCl (or other precursor sulfamoyl nonfluorohalide to the desired/indicated related derivative) resulted in formation of DMSF (or the desired/indicated related derivative) in at least about 85% yield, typically in at least about 90% yield, often at least about 95% yield, and more often at least about 99% yield.

In some embodiments, adding a catalyst is desirable. In particular and in some instances, DMSCl (or other precursor chlorinated sulfamoyl to the desired/indicated related derivative) is reacted with HF in the presence of a catalyst. The desired/indicated catalyst should be a Lewis acid. Suitable Lewis acids that can be used in processes of the present disclosure include salts of an alkaline metal, arsenic, antimony, bismuth, and zinc. In some embodiments, suitable catalysts for methods of the invention include, but are not limited to, Bi(III) compounds (such as BiCl₃, BiF₃) and Sb(III) compounds (such as SbCl₃, and SbF₃). When a catalyst is used, typically about 0.5 equivalent or less, often about 0.2 equivalent or less, and more often about 0.1 equivalent or less relative to the total initial amount of DMSCl (or other precursor sulfamoyl nonfluorohalide to the desired/indicated related derivative) is added to the reaction. The process of this aspect of the invention comprises: reacting DMSCl (or other precursor sulfamoyl nonfluorohalide to the desired/indicated related derivative) with HF under conditions sufficient to reflux HF and selectively removing hydrogen nonfluorohalide (e.g., HCl) that is formed in the reaction to produce the DMSF (or other desired/indicated related derivative) product.

In one particular embodiment, the reaction conditions comprise a pressure of about atmospheric pressure. In some embodiments, the reaction is conducted in a continuously stirred tank reactor with continuous DMSCl (or other precursor sulfamoyl nonfluorohalide to the desired/indicated related derivative) and HF feeds. In some instances, the crude product stream is distilled to recover purified DMSF (or the desired/indicated related derivative). Any unreacted DMSCl (or other precursor sulfamoyl nonfluorohalide to the desired/indicated related derivative) and HF that may be present can be recycled back into the reactor. It should be appreciated that in the example reaction shown below involving exchange of a nonfluorohalide (here, chlorine) with a fluorine atom, an equilibrium between forward and reverse reactions can limit the conversion to the desired exchanged product.

DMSCl+2HF [symbol] DMSF+2HF

According to the Le Chatelier's Principle, selective removal of the HCl product in this reaction would shift equilibrium to the right-hand side thereby producing more of the desired/indicated DMSF (or desired/indicated related derivative). It is noted that in one of the examples, the running the reaction of DMSCl with HF in a sealed vessel leads to incomplete reaction.

Differing instantiations of processes of the present disclosure can utilize either a closed vessel or an open reactor. In examples of using a closed vessel and a precursor sulfamoyl chloride, the HCl byproduct was allowed to release after 4 hours of rarefaction, and the closed vessel reaction was repeated one more time to give 100% conversion of starting material to the end product. In examples of using an open reactor and a precursor sulfamoyl chloride, the HCl byproduct was removed while preventing HF from escaping by condensing gaseous HF back into the reaction mixture. In particular, some instantiations involved boiling or distilling the volatile species HF and HCl from the reaction mixture and selectively condensing and returning HF back into the reaction mixture while allowing gaseous HCl to leave the reaction mixture. Alternatively, membrane separation, extraction, adsorption, ion exchange, and/or other separation method(s) can be used to selectively remove HCl from the reaction mixture.

As noted above, a catalyst can act to increase the equilibrium and/or the rate of reaction so that the reaction proceeds more quickly at a particular temperature. It should be appreciated, however, that the reaction does not require a catalyst to give acceptable results. In some instances, it was shown that the catalyst enhances reaction rate significantly.

As also noted above, processes of the present disclosure may be conducted in either a batchwise or continuous fashion. In an example batchwise approach, a reactor may be loaded with DMSCl (or other precursor sulfamoyl nonfluorohalide to the desired/indicated related derivative), HF, and optionally a catalyst, and then the HF may be refluxed, for example, at >20° C., until the HCl (or other hydrogen nonfluorohalogen) is completely removed. In practice, the boiling point temperature of the reaction mixture strongly depends on the amount of unreacted HF in the reactor, with higher HF concentrations giving lower reaction boiling points. Thus, to maintain a sufficiently high reaction temperature, HF may be added gradually during the reaction to prevent the amount of excess HF at any given time from being too high to achieve the desired reaction temperature.

HCl is a gas at room temperature with a normal atmospheric boiling point of −85° C. The reaction boiling point temperature can be used to monitor reaction progress. As HF is consumed, the reaction boiling point increases. Carefully metering of the HF feed rate can maintain a constant temperature and can also indicate the reaction rate. The reaction is completed when the feed rate drops to zero at the reaction temperature. In continuous operation, a continuously stirred tank reactor (CSTR) is advantageous, as it allows HF refluxing and continuous HCl removal. By design, a CSTR cannot operate at complete conversion, and, therefore, the product from the reactor is crude and has residual HF and DMSCl (or other precursor sulfamoyl nonfluorohalide to the desired/indicated related derivative).

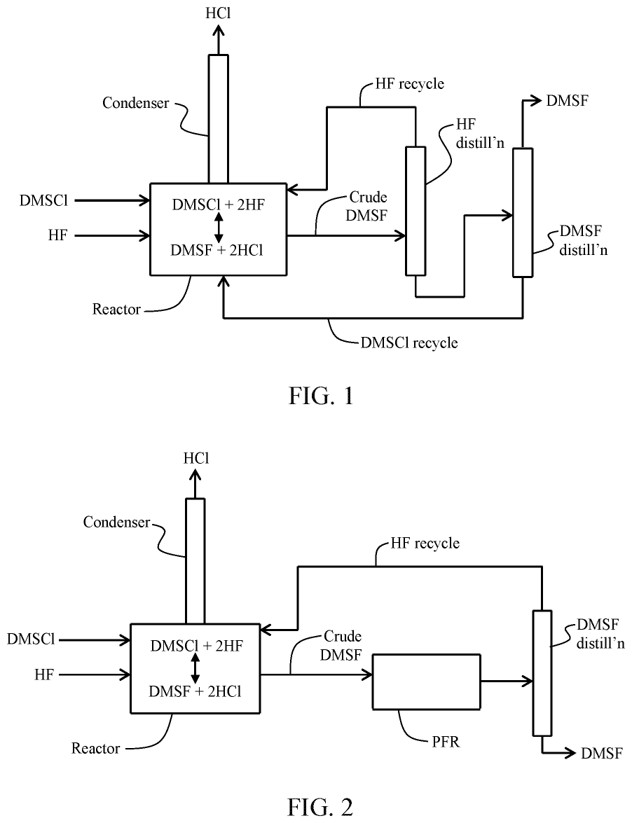

The crude DMSF (or related derivative) product, however, can be purified, for example, using two-stage distillation to remove volatile HF and the high-boiling DMSCl (or other precursor sulfamoyl nonfluorohalide to the desired/indicated related derivative). The recovered HF and DMSCl (or other precursor sulfamoyl nonfluorohalide to the desired/indicated related derivative) can be recycled back into the CSTR. See FIG. 1 for an example CSTR implementation. The second stage distillation is advantageously operated under vacuum (e.g., about 5 torr to about 60 torr) in order to avoid thermal degradation of the DMSF (or related derivative) product.

In another embodiment, a plug flow reactor (PFR) may follow the CSTR, wherein the unreacted DMSCl (or other precursor sulfamoyl nonfluorohalide to the desired/indicated related derivative) is completely converted to DMSF (or related derivative). An example of this configuration is shown in FIG. 2. In this configuration, a single distillation column or gas stripping column can be used to remove volatile HCl and recover HF. Again, the recovered HF can be recycled by returning it back to the CSTR.

Additional objects, advantages, and novel features of this invention will become apparent to those skilled in the art upon examination of the following examples thereof, which are not intended to be limiting. In the Examples below, procedures that are constructively reduced to practice are described in the present tense, and procedures that have been carried out in the laboratory are set forth in the past tense.

### EXAMPLES

### Example 1

To a dry 3.6-liter stainless steel (SS) cylinder (autoclave) having a ¼-inch SS ball valve and containing 200 grams of steel shot (to ensure mixing) was added 143.5 grams (1 mole) of N,N-dimethyl sulfamoyl chloride, and the cylinder was cooled to −78° C. using a dry ice-methanol bath for 30 minutes followed by evacuating the cylinder for 10 minutes. 60 grams of anhydrous HF (AHF) was transferred into the autoclave and allowed to warm up to room temperature. The autoclave was placed in an oven and heated to 90° C. and the contents allowed to react at 90° C. for 4 hours. Once the reaction was complete, the autoclave was allowed to cool to room temperature, at which point pressure was vented and scrubbed (70 psi max pressure developed). The autoclave was chilled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C. and the contents allowed to react at 90° C. for 4 hours. The autoclave was allowed to cool to room temperature, at which point pressure was vented and scrubbed (20 psi max pressure developed). The contents of the autoclave were poured into ice water, and the lower product phase was separated and treated with K₂CO₃ to neutralize any residual HF. The crude product was distilled at reduced pressure to yield 125 g of N,N-dimethyl sulfamoyl fluoride. The product was characterized by ¹H and ¹⁹F NMR. The reaction of this example is illustrated immediately below.

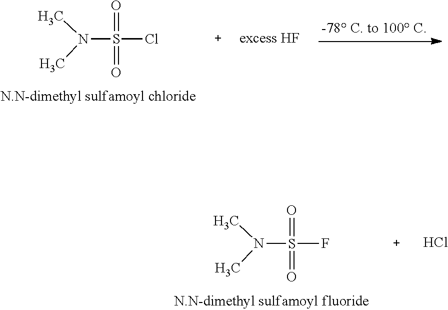

### Example 2

To a dry 3.6-liter SS cylinder (autoclave) having a ¼-inch SS ball valve and containing 200 grams of steel shot (to ensure mixing) was added 171.5 grams (1 mole) of N,N-diethyl sulfamoyl chloride, and the cylinder was cooled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C. and the contents allowed to react at 90° C. for 4 hours. Once the reaction was complete, the autoclave was allowed to cool to room temperature, at which point pressure was vented and scrubbed. The autoclave was chilled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C. and the contents allowed to react at 90° C. for 4 hours. The autoclave was allowed to cool to room temperature, at which point pressure was vented and scrubbed. Contents of the autoclave were poured into ice water, and the lower product phase was separated and treated with K₂CO₃ to neutralize any residual HF. The crude product was distilled at reduced pressure to yield 150 g of N,N-diethyl sulfamoyl fluoride. The product was characterized by ¹H and ¹⁹F NMR. The reaction of this example is illustrated immediately below.

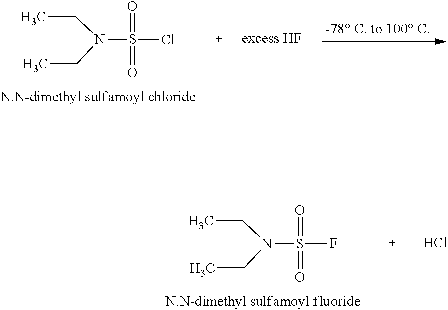

### Example 3

To a dry 3.6-liter SS cylinder (autoclave) having a ¼-inch SS ball valve and containing 200 grams of steel shot (to ensure mixing) was added 157.5 grams (1 mole) of N-ethyl N-methyl sulfamoyl chloride, and the cylinder was cooled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C., and the contents were allowed to react at 90° C. for 4 hours. Once the reaction was complete, the autoclave was allowed to cool to room temperature at which point pressure was vented and scrubbed. The autoclave was chilled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C., and the contents was allowed to react at 90° C. for 4 hours. The autoclave was allowed to cool to room temperature, at which point pressure was vented and scrubbed (20 psi (137.9 kPa) max pressure developed). Contents of the autoclave were poured into ice water, and the lower product phase was separated and treated with K₂CO₃ to neutralize any residual HF. The crude product was distilled at reduced pressure to yield 135 g of N-ethyl N-methyl sulfamoyl fluoride. The product was characterized by 1H and 19F NMR. The reaction of this example is illustrated immediately below

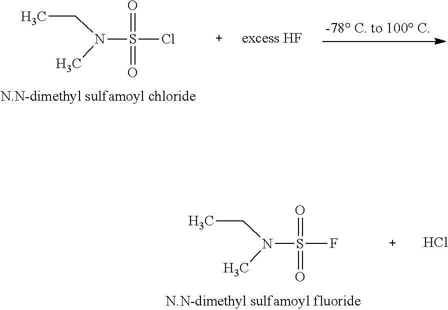

### Example 4

To a dry 3.6-liter SS cylinder (autoclave) having a ¼-inch SS ball valve and containing 200 grams steel shot (to ensure mixing) was added 169.5 grams (1 mole) of pyrrolidine sulfamoyl chloride, and the cylinder was cooled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C., and the contents was allowed to react at 90° C. for 4 hours. Once the reaction was complete, the autoclave was allowed to cool to room temperature, at which point pressure was vented and scrubbed. The autoclave was chilled to −78° C. using a dry ice-methanol bath for 30 minutes followed by evacuating the cylinder for 10 minutes. 60 grams of AHF was transferred into the autoclave and allowed to warm up to room temperature. The autoclave was placed in an oven and heated to 90° C., and the contents was allowed to react at 90° C. for 4 hours. The autoclave was allowed to cool to room temperature, at which point pressure was vented and scrubbed. The contents of the autoclave were poured into ice water, and the lower product phase was separated and treated with K₂CO₃ to neutralize any residual HF. The crude product was distilled at reduced pressure to yield 150 g of pyrrolidine sulfamoyl fluoride. The product was characterized by ¹H and ¹⁹F NMR. The reaction of this example is illustrated immediately below.

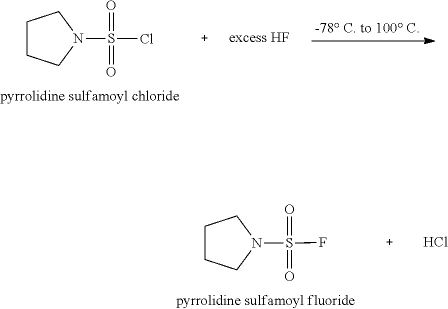 [SMILES: O=S(=O)(Cl)N1CCCC1 ; O=S(=O)(F)N1CCCC1 ; Cl]

### Example 5

To a dry 3.6-liter SS cylinder having a ¼-inch SS ball valve and containing 200 grams of steel shot (to ensure mixing) was added 203.5 grams (1 mole) of N,N-bis(2-methoxyethyl) sulfamoyl chloride, and the cylinder was cooled to −78° C. using a dry ice-methanol bath for 30 minutes followed by evacuating the cylinder for 10 minutes. 60 grams of AHF was transferred into the autoclave, and the contents was allowed to warm up to room temperature. The autoclave was placed in an oven and heated to 90° C., and the contents was allowed to react at 90° C. for 4 hours. Once the reaction was complete, the autoclave was allowed to cool to room temperature, at which point pressure was vented and scrubbed. The autoclave was chilled to −78° C. using dry ice-methanol bath for 30 minutes followed by evacuating the cylinder for 10 minutes. 60 grams of AHF was transferred into the autoclave, and the contents allowed to warm up to room temperature. The autoclave was placed in an oven and heated to 90° C., and the contents were allowed to react at 90° C. for 4 hours. The autoclave was allowed to cool to room temperature, at which point pressure was vented and scrubbed. The contents of the autoclave were poured into ice water, and the lower product phase was separated and treated with K₂CO₃ to neutralize any residual HF. The crude product was distilled at reduced pressure to yield 179 g of N,N-bis(2-methoxyethyl) sulfamoyl fluoride. The product was characterized by ¹H and ¹⁹F NMR. The reaction of this example is illustrated immediately below.

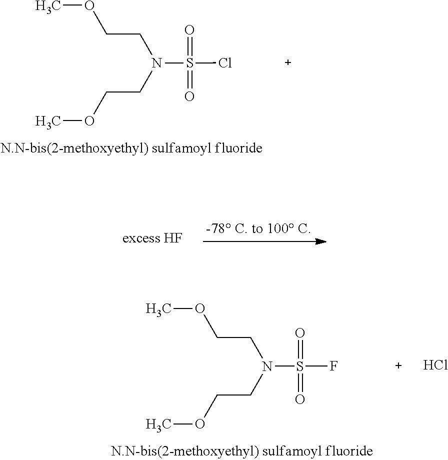

### Example 6

In a 150 ml perfluoroalkoxy alkane (PFA) reactor equipped with a PFA-coated thermocouple (to monitor reaction temperature), 35.87 g (0.25 mol) of N,N-dimethyl sulfamoyl chloride (DMSCl) was added. The reactor was connected to a condenser having a polytetrafluoroethylene (PTFE) vertical 60 mm long tube with an internal diameter of 12 mm. The outside of the condenser tube was jacketed with a vessel holding a mixture of dry ice and isopropanol. The top of the condenser was swept with dry argon, which carried gases from the top of the condenser to an alkaline scrubber before venting. An inlet port to the reactor provided means to feed gaseous AHF into the system, which would condense in the condenser and drip into the reactor. The reactor was laced in an oil bath. A total 10 g (0.5 mol) of AHF was used to convert the DMSCl to DMSF. The HF was added in increments. The first addition was 5 g (0.25 mol) HF, and the solution boiled at 40° C. and was refluxing. Ambient pressure was 85 kPa. After 20 minutes, the reactor was cooled in an ice bath, opened under argon, and 1.5 g BiF₃ was added as a catalyst, after which the reactor was resealed and reheated. Boiling and refluxing was observed at 40° C., and the reaction temperature slowly increased to 85° C. over 2 hours. Another 5 g of anhydrous HF was added, which dropped the boiling point to 70° C. and slowly heated to 85° C. in 30 minutes. The condenser was heated to room temperature, and excess HF was allowed to boil from the reactor at 85° C. for 1.5 hour. The reactor was cooled to room temperature and product was isolated by distillation at reduced pressure to yield N,N-dimethyl sulfamoyl fluoride (DMSF) in 95% yield. The product was characterized by 1H and 19F NMR. The reaction of this example is illustrated immediately below.

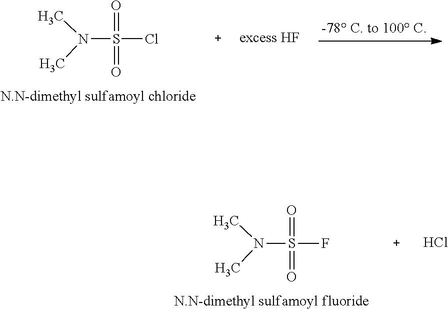

### Example 7

Using the same PFA reactor system as described in Example 6, (0.25 mol) of N-ethyl-N-methyl sulfamoyl chloride was charged to the reactor. No bismuth material or PTFE boiling stones were added. The reactor was heated to 85° C., and a total 10 g (0.5 mole) of HF was added portion-wise while trying to maintain the reaction temperature near 85° C. The reactor temperature ranged between 60° C. and 85° C. After multiple small HF additions over the course of 5 hours, the temperature stabilized at 85° C. The reactor was cooled to room temperature and the product was isolated by distillation at reduced pressure to yield N-ethyl-N-methyl sulfamoyl fluoride in 96% yield. The product was characterized by ¹H and ¹⁹F NMR. The reaction of this example is illustrated immediately below.

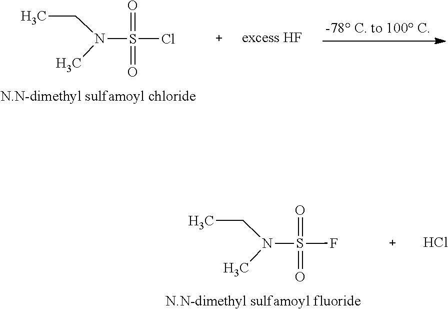

Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve aspects of the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

1. A method of producing a sulfamoyl fluoride compound of the formula F—SO₂—NR₂, wherein either 1) each R is, independently, a linear or branched alkyl, alkenyl, or alkynyl group containing 1 to 12 carbon atoms or 2) R₂ forms a cyclic amine with the N, the method comprising:
adding a sulfamoyl nonfluorohalide of the formula X—SO₂—NR₂ and hydrogen fluoride (HF) to a reaction chamber of a reaction apparatus, wherein X is selected from the group consisting of chlorine (Cl), bromine (Br), and iodine (I);
providing conditions sufficient to support a reaction between the sulfamoyl nonfluorohalide and the HF that forms the sulfamoyl fluoride compound and an HX byproduct; and
selectively removing at least some of the HX byproduct so as to yield the sulfamoyl fluoride compound.

2. The method of claim 1, wherein providing conditions sufficient to support a reaction between the sulfamoyl nonfluorohalide and the HF include providing HF refluxing conditions.

3. (canceled)

4. The method of claim 1, wherein providing conditions sufficient to support a reaction between the sulfamoyl nonfluorohalide and the HF include heating the reaction chamber to a temperature of at least about 20° C.

5. (canceled)

6. (canceled)

7. The method of claim 1, wherein adding the sulfamoyl nonfluorohalide and the HF includes adding the HF batchwise.

8. (canceled)

9. (canceled)

10. The method of claim 1, wherein adding the sulfamoyl nonfluorohalide and the HF includes adding the HF at least about 1.5 equivalents relative to the sulfamoyl nonfluorohalide.

11. The method of claim 1, wherein adding the sulfamoyl nonfluorohalide and the HF includes adding the HF at least about 2 equivalents relative to the sulfamoyl nonfluorohalide.

12. The method of claim 1, wherein the reaction has a yield of the sulfamoyl fluoride of at least about 85%.

13. The method of claim 1, wherein the reaction has a yield of the sulfamoyl fluoride of at least about 95%.

14. The method of claim 1, wherein the reaction has a yield of the sulfamoyl fluoride of at least about 99%.

15. The method of claim 1, wherein the reaction occurs in the presence of a catalyst.

16. The method of claim 15, wherein said catalyst comprises a Bi(III) compound.

17. (canceled)

18. The method of claim 15, wherein about 0.5 equivalents or less of the catalyst, relative to a total amount of the sulfamoyl nonfluorohalide, is added to the reaction.

19. (canceled)

20. The method of claim 1, wherein each R is selected, independently, from the group consisting of a methyl group, an ethyl group, and a methoxyethyl group.

21. The method of claim 1, wherein each R is H₃C—.

22. The method of claim 1, wherein each R is H₃C—CH₂—.

23. (canceled)

24. The method of claim 1, wherein each R is a 2-methoxyethyl group.

25. (canceled)

26. The method of claim 1, wherein the HX byproduct is in gaseous form, and selectively removing at least a portion of the HX byproduct includes condensing the HX byproduct.

27. (canceled)

28. The method of claim 1, further comprising recycling the condensed HF back into the reaction.

29. The method of claim 1, wherein adding a sulfamoyl nonfluorohalide and HF to a reaction chamber includes adding a sulfamoyl nonfluorohalide and HF to a continuously stirred tank reactor operated so as to provide HF refluxing and continuous removal of the HX byproduct.

30. The method of claim 29, wherein a portion of the sulfamoyl nonfluorohalide is unreacted, and the reaction apparatus includes, downstream of the continuously stirred tank reactor, a plug flow reactor that converts the unreacted sulfamoyl nonfluorohalide to the sulfamoyl fluoride.
